(19) United States
(12) Patent Application Publication
(10) Pub. No.: US 2008/0171892 A1
(43) Pub. Date: Jul. 17, 2008

(54) Method for Producing Optically Active 3-Phenylpropionic Acid Derivatives and Follow-On Products of the Latter

(75) Inventors: Frank Hettche, Weinheim (DE); Martin Volkert, Ludwigshafen (DE); Christoph Jakel, Limburgerhof (DE); Oliver Bey, Niederkirchen (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/908,808

(22) PCT Filed: Mar. 16, 2006
(86) PCT No.: PCT/EP2006/002435

§ 371 Date: Sep. 17, 2007

(30) Foreign Application Priority Data
Mar. 17, 2005 (DE) 10 2005 012 408.9
Jun. 23, 2005 (DE) 10 2005 029 228.3
Jan. 5, 2006 (DE) 10 2006 000 839.1

(51) Int. Cl. C07C 67/00; C07C 17/16

(52) U.S. Cl. 560/104; 570/201

(57) ABSTRACT

The present invention relates to a method for preparing optically active 3-phenylpropionic acid derivatives, to optically active 1-chloro-3-phenylpropane derivatives obtainable therefrom and to optically active intermediates obtained thereby.

The present invention relates to a method for preparing optically active 3-phenylpropionic acid derivatives, to optically active 1-chloro-3-phenylpropane derivatives obtainable therefrom and to optically active intermediates obtained thereby.

Asymmetric synthesis, i.e. reactions in which a chiral grouping is generated from a prochiral one so that the stereoisomeric products (enantiomers or diastereomers) result in unequal amounts, has become immensely important especially in the pharmaceutical industry sector because frequently only one particular optically active isomer has therapeutic activity. The same also applies to the following compound which is referred to as synthon A

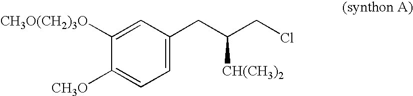

which is an important intermediate in the preparation of the renin inhibitor aliskiren (SPP100). Aliskiren is a highly active and selective renin inhibitor and as such an important potential active pharmaceutical ingredient for the treatment of high blood pressure and related cardiovascular disorders (J. M. Wood et al., Biochemical and Biophysical Research Communications 308 (2003) 698-705). There is thus a great need for efficient synthetic routes to systems of the type of synthon A and its optical antipodes.

WO 02/02500 and Adv. Synth. Catal. 2003, 345, 160-164 describe the synthesis of (R)-2-alkyl-3-phenylpropionic acids as intermediates in the preparation of synthon A by asymmetric hydrogenation of the corresponding trans-acrylic acids as shown in the following scheme

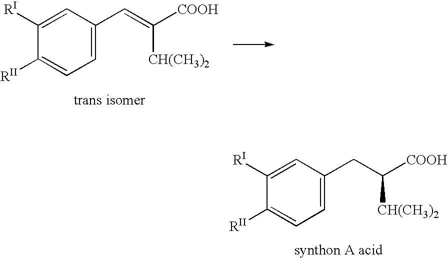

One disadvantage of this method is the elaborate preparation of the trans isomer by repeated extraction and crystallization. In addition, the catalyst which is employed for the enantioselective hydrogenation, and which is based on a phosphine ligand with phenylferrocenyl backbone, allows only a low substrate/catalyst ratio (s/c=5700) with only 95% ee, so that correspondingly large amounts of catalyst must be employed, making the method economically disadvantageous.

It is therefore an object of the present invention to provide a novel method for preparing optically active 3-phenylpropionic acid derivatives and their follow-on or resultant products, especially synthon A, which permits efficient and cost-effective industrial synthesis. It is intended in this connection in particular for it to be possible to employ a cis/trans isomer mixture of 3-phenylacrylic acid derivatives as intermediates. It is further intended to achieve a high optical yield (≧98% ee) with substrate/catalyst ratios which are as high as possible, i.e. small amounts of catalyst (s/c≧10 000/l).

This object is achieved by a method for preparing optically active compounds of the general formula I

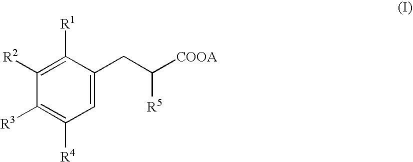

in which
R¹, R², R³ and R⁴ are independently of one another hydrogen, C₁-C₆-alkyl, halo-C₁-C₆-alkyl, hydroxy-C₁-C₆-alkyl, C₁-C₆-alkoxy, hydroxy-C₁-C₆-alkoxy, C₁-C₆-alkoxy-C₁-C₆-alkyl, hydroxy-C₁-C₆-alkoxy-C₁-C₆-alkyl, C₁-C₆-alkoxy-C₁-C₆-alkoxy or hydroxy-C₁-C₆-alkoxy-C₁-C₆-alkoxy,
R⁵ is C₁-C₆-alkyl, C₅-C₈-cycloalkyl, phenyl or benzyl, and
A is hydrogen or a cation equivalent, in which
   the cis isomer or a cis/trans isomer mixture of compounds of the general formula II

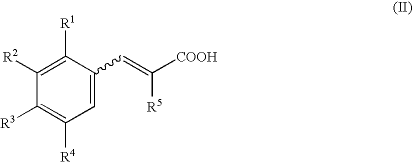

in which R¹ to R⁵ have the aforementioned meanings, is subjected to an enantioselective hydrogenation in the presence of a chiral hydrogenation catalyst to obtain a mixture of enantiomers enriched in one enantiomer,
the mixture of enantiomers obtained in the hydrogenation is subjected, for further enantiomer enrichment, to a crystallization by adding a basic salt former in a solvent, and the solid which is formed thereby and is enriched in one stereoisomer is isolated, and
if appropriate the isolated isomer is subjected to a protonation or a cation exchange to obtain the optically active compound of the formula I.

“Chiral compounds” are in the context of the present invention compounds having at least one chirality center (i.e. at least one asymmetric atom, in particular at least one asymmetric C atom or P atom), with chirality axis, chirality plane or helical twist. The term “chiral catalyst” comprises catalysts which have at least one chiral ligand.

“Achiral compounds” are compounds which are not chiral.

A “prochiral compound” means a compound having at least one prochiral center. “Asymmetric synthesis” refers to a reaction in which a compound with at least one chirality center, one chirality axis, chirality plane or helical twist is generated from a compound with at least one prochiral center, with the stereoisomeric products resulting in unequal amounts.

“Stereoisomers” are compounds of identical constitution but different arrangement of atoms in three-dimensional space.

“Enantiomers” are stereoisomers which are related to one another as image to mirror image. The “enantiomeric excess” (ee) achieved in an asymmetric synthesis results from the following formula: ee[%]=(R—S)/(R—S)×100. R and S are the descriptors of the CIP system for the two enantiomers and represent the absolute configuration at the asymmetric atom. The enantiopure compound (ee=100%) is also referred to as “homochiral compound”.

The method of the invention leads to products which are enriched in a particular stereoisomer. The “enantiomeric excess” (ee) achieved is generally at least 98%.

“Diastereomers” are stereoisomers which are not enantiomers of one another.

The term “alkyl” hereinafter comprises straight-chain and branched alkyl groups. These are preferably straight-chain or branched C₁-C₂₀-alkyl, more preferably C₁-C₁₂-alkyl, particularly preferably C₁-C₈-alkyl and very particularly preferably C₁-C₆-alkyl groups. Examples of alkyl groups are in particular methyl, ethyl, propyl, isopropyl, n-butyl, 2-butyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The term “alkyl” also comprises substituted alkyl groups which may generally have 1, 2, 3, 4 or 5, preferably 1, 2 or 3 and particularly preferably 17 substituents selected from the groups cycloalkyl, aryl, hetaryl, halogen, NE¹E², NE¹E²E³⁺, COOH, carboxylate, —SO₃H and sulfonate.

The term “alkylene” for the purposes of the present invention stands for straight-chain or branched alkanediyl groups having preferably 1 to 6, in particular 1 to 4, carbon atoms. These include methylene (—CH₂—), ethylene (—CH₂—CH₂—), n-propylene (—CH₂—CH₂—CH₂—), isopropylene (—CH₂—CH(CH₃)—) etc.

The term “cycloalkyl” comprises for the purposes of the present invention unsubstituted and substituted cycloalkyl groups, preferably C₃-C₈-cycloalkyl groups such as cyclopentyl, cyclohexyl or cycloheptyl, which in the event of substitution may generally have 1, 2, 3, 4 or 5, preferably 1, 2 or 3 and particularly preferably 1, substituents, preferably selected from the substituents mentioned for alkyl.

The term “heterocycloalkyl” for the purposes of the present invention comprises saturated cycloaliphatic groups having in general 4 to 7, preferably 5 or 6, ring atoms in which 1 or 2 of the ring carbon atoms are replaced by heteroatoms, preferably selected from the elements oxygen, nitrogen and sulfur, and which may optionally be substituted, where in the event of substitution these heterocycloaliphatic groups may have 1, 2 or 3, preferably 1 or 2, particularly preferably 1, substituents selected from alkyl, aryl, COOR^{f}, COO⁻M⁺ and NE¹E², preferably alkyl. Examples which may be mentioned of such heterocycloaliphatic groups are pyrrolidinyl, piperidinyl, 2,2,6,6-tetramethylpiperidinyl, imidazolidinyl, pyrazolidinyl, oxazolidinyl, morpholidinyl, thiazolidinyl, isothiazolidinyl, isoxazolidinyl, piperazinyl, tetrahydrothiophenyl, tetrahydrofuranyl, tetrahydropyranyl, dioxanyl.

The term “aryl” comprises for the purposes of the present invention unsubstituted and substituted aryl groups and stands preferably for phenyl, tolyl, xylyl, mesityl, naphthyl, fluorenyl, anthracenyl, phenanthrenyl or naphthacenyl, particularly preferably for phenyl or naphthyl, where these aryl groups may in the event of substitution have in general 1, 2, 3, 4 or 5, preferably 1, 2 or 3 and particularly preferably 1, substituents selected from the groups alkyl, alkoxy, carboxyl, carboxylate, —SO₃H, sulfonate, NE¹E², alkylene-NE¹E², nitro, cyano or halogen.

The term “hetaryl” comprises for the purposes of the present invention unsubstituted or substituted heterocycloaromatic groups, preferably the groups pyridinyl, quinolinyl, acridinyl, pyridazinyl, pyrimidinyl, pyrazinyl, pyrrolyl, imidazolyl, pyrazolyl, indolyl, purinyl, indazolyl, benzotriazolyl, 1,2,3-triazolyl, 1,3,4-triazolyl and carbazolyl, where these heterocycloaromatic groups may in the event of substitution have in general 1, 2 or 3 substituents selected from the groups alkyl, alkoxy, acyl, carboxyl, carboxylate, —SO₃H, sulfonate, NE¹E², alkylene-NE¹E² or halogen.

The above explanations of the terms “alkyl”, “cycloalkyl”, “aryl”, “heterocycloalkyl” and “hetaryl” apply correspondingly to the terms “alkoxy”, “cycloalkoxy”, “aryloxy”, “heterocycloalkoxy” and “hetaryloxy”.

The term “acyl” for the purposes of the present invention stands for alkanoyl or aroyl groups having in general 2 to 11, preferably 2 to 8, carbon atoms, for example the acetyl, propanoyl, butanoyl, pentanoyl, hexanoyl, heptanoyl, 2-ethylhexanoyl, 2-propylheptanoyl, benzoyl, or naphthoyl group.

The groups NE¹E² preferably stand for N,N-dimethylamino, N-ethyl-N-methylamino, N,N-diethylamino, N,N-dipropylamino, N,N-diisopropylamino, N,N-di-n-butylamino, N,N-di-t-butylamino, N,N-dicyclohexylamino or N,N-diphenylamino.

Halogen stands for fluorine, chlorine, bromine and iodine, preferably for fluorine, chlorine and bromine.

A cation equivalent means a singly charged cation or the fraction of a multiply charged cation which corresponds to a positive single charge. Preferably alkali metal, in particular Na⁺, K⁺, Li⁺, ions or onium ions such as ammonium, mono-, di-, tri-, tetraalkylammonium, phosphonium, tetraalkylphosphonium or tetraarylphosphonium ions are used.

The radicals R¹, R², R³ and R⁴ are preferably independently of one another hydrogen, C₁-C₄-alkyl such as methyl, ethyl, n-propyl, isopropyl, n-butyl and tert-butyl, C₁-C₄-alkoxy such as methoxy, ethoxy, n-propyloxy or isopropyloxy, or C₁-C₄-alkoxy-C₁-C₄-alkoxy such as methoxyethoxy, ethoxyethoxy, methoxy-n-propyloxy, ethoxy-n-propyloxy.

Preferably R¹ and R⁴ are hydrogen, and R² and R³ are selected independently of one another from the aforementioned suitable and preferred radicals different from hydrogen.

Preferably R² is methoxy-n-propyloxy and R³ is methoxy.

The radical R⁵ is preferably C₁-C₆-alkyl, preferably branched C₃-C₆-alkyl and in particular isopropyl.

A is particularly preferably hydrogen or a cation derived from ammonia, primary amines, alkali metals and alkaline earth metals. A is in particular H⁺, NH₄⁺ or Li⁺.

The method of the invention serves in a specific embodiment for preparing “synthon A acid” of the following formula

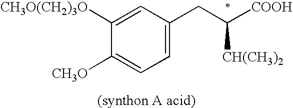

in high optical purity, in particular with an ee of at least 98% (*=stereocenter).

The method of the invention makes it possible to prepare optically active compounds of the general formula I as described above starting from the cis isomer or preferably a cis/trans isomer mixture of compounds of the general formula II. A cis/trans isomer mixture of compounds of the general formula II which comprises the cis isomer in an amount of at least 40%, preferably in excess, is preferably employed. The isomer mixture employed for the hydrogenation preferably then comprises cis isomer in an amount of at least 50% by weight, particularly preferably at least 60% by weight and in particular at least 70% by weight, based on the total weight of cis isomer and trans isomer.

It is a characteristic feature of the method of the invention that the isomer mixture of compounds of the general formula II employed for the enantioselective hydrogenation also comprises the trans isomer in non-negligible amounts. The method thus advantageously makes it possible to prepare optically active compounds of the general formula I starting from cis/trans isomer mixtures of compounds of the general formula II as are obtainable for example from precursor compounds by conventional 1,2 elimination, preferably with a certain cis stereoselectivity. The cis/trans isomer mixture of compounds of the general formula II employed for the hydrogenation preferably comprises the trans isomer in an amount of at least 1% by weight, particularly preferably at least 5% by weight and in particular at least 10% by weight, based on the total weight of cis isomer and trans isomer.

The method of the invention advantageously makes it possible to prepare the compounds of the formula I starting from cis/trans isomer mixtures in technical purity grades. It is thus generally possible to dispense with elaborate purification steps before the hydrogenation. The cis/trans isomer mixture compositions employed preferably comprise at least 80% by weight, particularly preferably at least 85% by weight, of cis and trans isomers based on the total weight of the compositions. Examples of further components present are solvents, and precursors, intermediates and byproducts from preceding reaction stages.

Preferably employed for the hydrogenation is a chiral hydrogenation catalyst which is able to hydrogenate the cis/trans isomer mixture employed with preference for the isomer whose absolute configuration corresponds to the (R) isomer of synthon A acid. A particularly high ee at the stage of the asymmetric hydrogenation is preferred, but is not decisive on its own, because a further enantiomeric enrichment takes place according to the method of the invention in the subsequent crystallization step. However, it has surprisingly been found that it is possible with the chiral hydrogenation catalysts described hereinafter and based on planar-chiral bisphosphanes with cyclophane backbone to hydrogenate both the cis isomer and the trans isomer in high optical purity to the desired enantiomer, i.e. with ee values of in each case at least 50% (e.g. at least 70%). When cis/trans isomer mixtures with a cis content of at least 70% by weight (based on the total weight of cis isomer and trans isomer) are employed, generally ee values of at least 80% are achieved, and when the cis content is 100% generally ee values of at least 90% are achieved.

It is therefore preferred to employ as catalyst for the hydrogenation a transition metal complex which comprises as ligand at least one compound of the formula

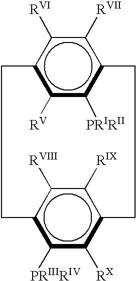

in which
R^{I}, R^{II}, R^{III} and R^{IV} are independently of one another alkyl, cycloalkyl, heterocycloalkyl, aryl or hetaryl, and
R^{V}, R^{VI}, R^{VII}, R^{VIII}, R^{IX} and R^{X} are independently of one another hydrogen, alkyl, alkylene-OH, alkylene-NE¹E², alkylene-SH, alkylene-OSiE³E⁴, cycloalkyl, heterocycloalkyl, aryl, hetaryl, OH, SH, polyalkylene oxide, polyalkyleneimine, alkoxy, halogen, COOH, carboxylate, SO₃H, sulfonate, NE¹E², nitro, alkoxycarbonyl, acyl or cyano, in which E¹, E², E³ and E⁴ are each identical or different radicals selected from hydrogen, alkyl, cycloalkyl, aryl and alkylaryl.

The radicals R^{I}, R^{II}, R^{III} and R^{IV} bonded to the phosphorus atoms are preferably selected independently of one another from unsubstituted and substituted aryl radicals. Preference is given to phenyl radicals which may have 1, 2, 3 or 4, preferably 1, 2, or 3, in particular 1 or 2, substituents which are preferably selected from alkyl, alkoxy, halogen, SO₃H, sulfonate, NE¹E², alkylene-NE¹E², trifluoromethyl, nitro, carboxyl, alkoxycarbonyl, acyl and cyano. For the substituents of the phenyls, alkyl is preferably C₁-C₄-alkyl and in particular methyl, ethyl, isopropyl and tert-butyl, alkoxy is preferably C₁-C₄-alkoxy and in particular methoxy, alkoxycarbonyl is preferably C₁-C₄-alkoxycarbonyl. The radicals R^{I}, R^{II}, R^{III} and R^{IV} are particularly preferably selected from phenyl, tolyl, methoxyphenyl, methoxyxylyl or xylyl, in particular from phenyl or xylyl. R^{I} to R^{IV} are preferably all phenyl or all tolyl or all methoxyphenyl or all xylyl or all methoxyxylyl. The tolyl radicals have the methyl group preferably in position 4 relative to the phosphorus atom. The methoxyphenyl radicals preferably have the methoxy group in position 4 relative to the phosphorus atom. The xylyl radicals preferably have the methyl groups in positions 3 and 5 relative to the phosphorus atom. The methoxyxylyl radicals preferably have the methoxy group in position 4 and the methyl groups preferably in positions 3 and 5 relative to the phosphorus atom.

It is preferred for at least one of the radicals R^{V}, R^{VI} and R^{VII} and/or one of the radicals R^{VIII}, R^{IX} and R^{X} to be a radical different from hydrogen, and for the other radicals to be hydrogen. The radical(s) different from hydrogen is/are preferably selected from C₁-C₆-alkyl, C₁-C₄-alkylene-OH, C₁-C₄-alkylene-OSi(C₁-C₄-alkyl)₂, C₁-C₄-alkoxy, C₁-C₄-alkylene-OC(alkyl)₃ and C₁-C₄-alkylene-OC(aryl)₃.

In a preferred embodiment, the radicals R^{V} to R^{X} are all hydrogen. In a further referred embodiment, one of the radicals R^{V}, R^{VI} and R^{VII} and/or one of the radicals R^{VIII}, R^{IX} and R^{X} is selected from the radicals of the formulae CH₂OSi(CH(CH₃)₂)₃, CH₂OH, OCH₃, CH₂OC(CH₃)₃ and CH₂OC(C₆H₅)₃, in particular from the radicals of the formulae CH₂OSi(CH(CH₃)₂)₃, CH₂OH, OCH₃ and CH₂OC(C₆H₅)₃.

The ligands particularly preferred as planar-chiral bisphosphane ligands with cyclophane backbone are those of the following formulae

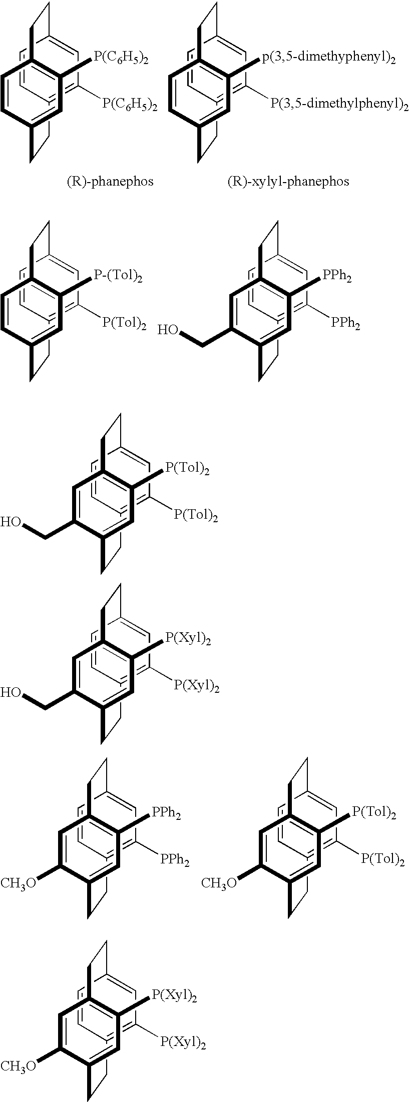

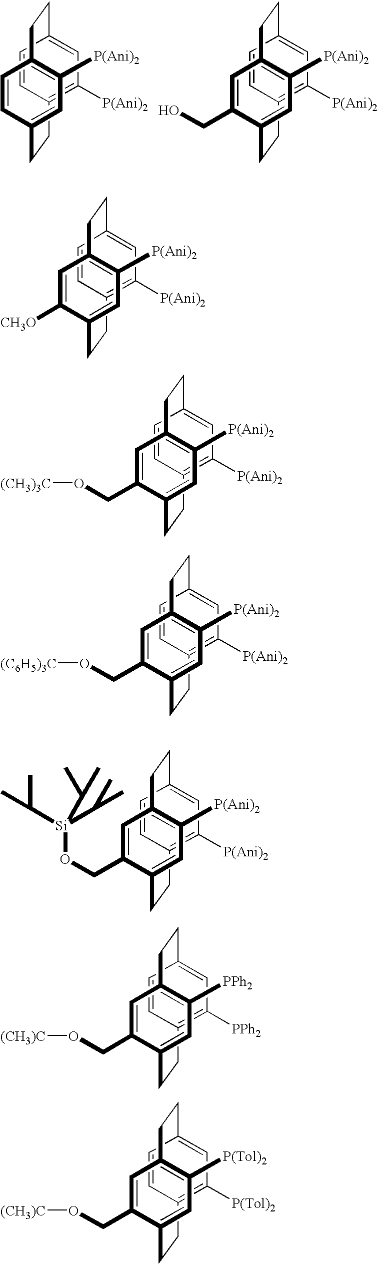

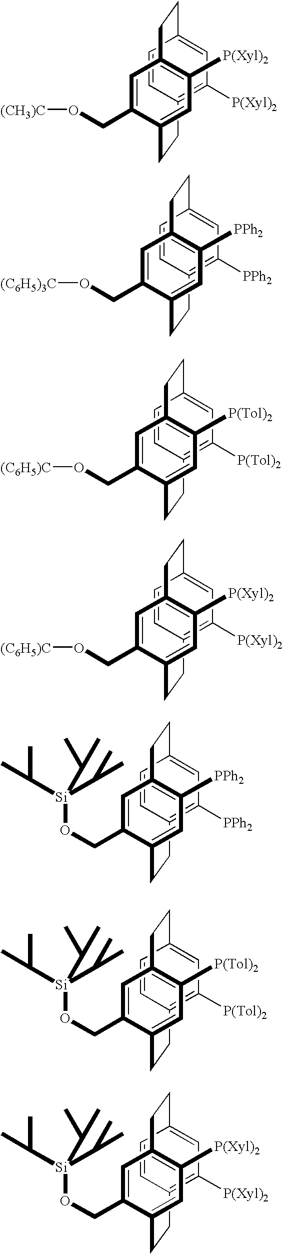

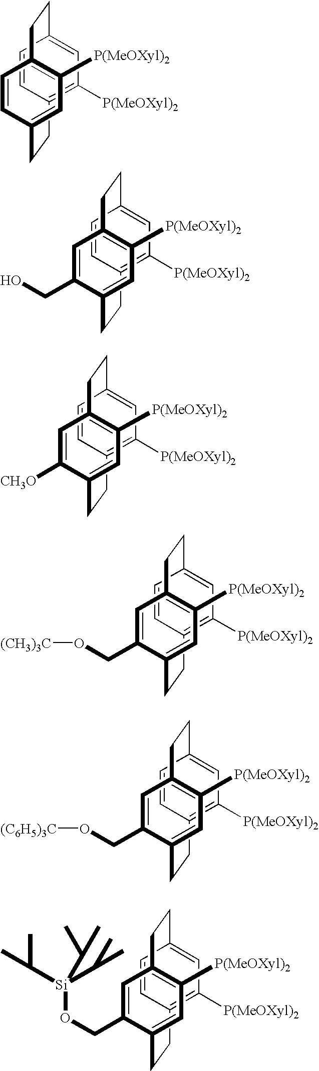

Ph=phenyl, Tol=4-methylphenyl, Xyl=3,5-dimethylphenyl, Ani=4-methoxyphenyl, MeOXyl=3,5-dimethyl-4-methoxyphenyl

Suitable chiral paracyclophanephosphines are known to the skilled worker and commercially available for example from Johnson Matthey Catalysts,

Assignment of the chiral descriptor “R” to the depicted ligands took place in accordance with P. J. Pye and K. Rossen, Tetrahedron: Asymmetry 9 (1998), pp. 539-541 and corresponds to the commercial designation of these ligands.

It is preferred to employ for the enantioselective hydrogenation a complex of a metal of group VIII of the periodic table having at least one of the aforementioned planar-chiral bisphosphane compounds with cyclophane backbone as ligand. The transition metal is preferably selected from Pd, Pt, Ru, Rh, Ni and Ir. Catalysts based on Rh, Ru and Ir are particularly preferred. Rh catalysts are particularly preferred.

Phosphine-metal complexes can be obtained in a manner known to the skilled worker (e.g. Uson, Inorg. Chim. Acta 73, 275 1983, EP-A-0 158 875, EP-A-437 690) by reacting the phosphines with complexes of the metals which comprise labile or hemilabile ligands. Sources of metals which can be used in this connection are complexes such as, for instance, Pd₂(dibenzylideneacetone)₃, Pd(Oac)₂, [Rh(COD)Cl]₂, [Rh(COD)₂)]X, Rh(acac)(CO)₂, RuCl₂(COD), Ru(COD)(methallyl)₂, Ru(Ar)Cl₂, Ar=aryl, both unsubstituted and substituted, [Ir(COD)Cl)₂, [Ir(COD)₂]X, Ni(allyl)X. Instead of COD (=1,5-cyclooctadiene) it is also possible to use NBD (=norbomadiene). Preference is given to [Rh(COD)Cl]₂, [Rh(COD)₂)]X, Rh(acac)(CO)₂, RuCl₂(COD), Ru(COD)(methallyl)₂, Ru(Ar)Cl₂, Ar=aryl, both unsubstituted and substituted, [Ir(COD)Cl]₂ and [Ir(COD)₂]X, and the corresponding systems with NBD replacing COD. [Rh(COD)₂)]X and [Rh(NBD)₂)]X are particularly preferred.

X can be ally anion known to the skilled worker to be generally useful in asymmetric synthesis. Examples of X are halogens such as Cl⁻, Br⁻, I⁻, BF₄⁻, ClO₄⁻, SbF₆⁻, PF₆⁻, CF₃SO₃⁻, BAr₄⁻. X is preferably BF₄⁻, CF₃SO₃⁻, SbF₆⁻, ClO₄⁻, in particular BF₄⁻ and CF₃SO₃⁻.

The phosphine-metal complexes can, as the skilled worker is aware, either be generated in situ in the reaction vessel before the actual hydrogenation reaction, or else be generated separately, isolated and subsequently employed. It is possible in this connection for at least one solvent molecule to undergo addition to the phosphine-metal complex. Common solvents (e.g. methanol, diethyl ether, dichloromethane) for preparing the complexes are known to the skilled worker.

As the skilled worker is aware, the phosphine-metal or phosphine-metal-solvent complexes are precatalysts still having at least one labile or hemilabile ligand, from which the actual catalyst is generated under the hydrogenation conditions.

Solvents suitable for the hydrogenation reaction are all solvents known to the skilled worker for asymmetric hydrogenation. Preferred solvents are lower alkyl alcohols such as methanol, ethanol, isopropanol, and toluene, THF, ethyl acetate. Methanol is particularly preferably employed as solvent in the method of the invention.

The hydrogenation of the invention is generally carried out at a temperature of from −20 to 200° C., preferably at from 0 to 150° C. and particularly preferably at from 20 to 120° C.

The hydrogen pressure may be varied in a wide range between 0.1 bar and 325 bar for the hydrogenation method of the invention. Very good results are obtained in a pressure range from 1 to 300 bar, preferably 5 to 250 bar.

The method of the invention preferably makes the enantioselective hydrogenation possible with substrate/catalyst ratios (s/c) of at least 1000:1, particularly preferably at least 10 000:1 and in particular at least 30 000:1. It is advantageous in this connection that even with substrate/catalyst ratios of 30 000:1 (when a cis/trans isomer mixture which comprises at least 70% cis isomer based on the total weight of cis isomer and trans isomer is employed) ee values of at least 80% are achieved. This is a crucial advantage over the hydrogenation catalysts employed in known methods.

The hydrogenation catalysts (or precatalysts) described above can also be immobilized in a suitable manner, e.g. by attachment via functional groups suitable as anchor groups, adsorption, grafting, etc., to a suitable support, e.g. made of glass, silica gel, synthetic resins, polymeric supports, etc. They are then also suitable for use as solid-phase catalysts. It is advantageously possible by these methods to reduce the catalyst consumption further. The catalysts described above are also suitable for a continuous reaction process, e.g. after immobilization as described above, in the form of solid-phase catalysts.

In a preferred embodiment, the hydrogenation takes place continuously. Continuous hydrogenation can take place in one or, preferably, in a plurality of reaction zones. A plurality of reaction zones can be formed by a plurality of reactors or by spatially different regions within one reactor. If a plurality of reactors is employed, the reactors may in each case be identical or different. They may in each case have identical or different mixing characteristics and/or be subdivided one or more times by internals. The reactors can be connected together as desired, e.g. in parallel or in series.

Suitable pressure-resistant reactors for hydrogenation are known to the skilled worker. These include the generally customary reactors for gas-liquid reactions, such as, for example, tube reactors, tube bundle reactors, stirred vessels, gas circulation reactors, bubble columns, etc., which may optionally be packed or subdivided by internals.

A preferred method for continuous hydrogenation is one in which
i) a mixture of isomers of compounds of the general formula II and hydrogen are fed into a first reaction zone and reacted in the presence of a chiral hydrogenation catalyst as far as partial conversion,
ii) a stream is taken from the first reaction zone and hydrogenated in at least one further reaction zone.

In a first preferred embodiment, the reactor employed for carrying out the aforementioned cascaded continuous hydrogenation method has two or more than two reaction zones which are established by internals. These internals may be for example perforated plates, random packings, ordered packings or combinations thereof. In a second preferred embodiment, the reaction system employed for carrying out the aforementioned cascaded continuous hydrogenation method consists of two reactors connected in series.

The temperature in the hydrogenation is generally in a range from about 10 to 200° C., preferably 20 to 150° C., in all reaction zones. It is possible if desired to set a different, preferably a higher, temperature in the second reaction zone than in the first reaction zone, or a higher temperature in each subsequent reaction zone than in a preceding reaction zone, e.g. in order to achieve maximum conversion in the hydrogenation. The reaction is carried out in all reaction zones preferably with a hydrogen pressure in a range from about 1 to 300 bar, preferably 5 to 250 bar. It is possible if desired to set a different, e.g. a higher, hydrogen pressure in the second or a subsequent reaction zone than in the first or a preceding reaction zone.

The reactor volume and/or the holdup time in the first reaction zone are chosen so that in general at least about 10% of the isomer mixture fed in are reacted. The conversion in the first reaction zone, based on the isomer mixture fed in, is preferably at least 80%.

To remove the heat of reaction produced in the exothermic hydrogenation, the first and/or the subsequent reaction zone(s) can be provided with a cooling device. Removal of the heat of reaction can take place by cooling an external circulation stream or by internal cooling in at least one of the reaction zones. It is possible to employ for the internal cooling the devices customary for this purpose, generally hollow modules such as Field tubes, coiled tubes, heat exchanger plates, etc. If the reaction mixture hydrogenated in the second or a subsequent reaction zone contains such small proportions of hydrogenatable compounds that the heat evolved in the reaction is insufficient to maintain the desired temperature in the reaction zone, heating of the second or a subsequent reaction zone may also be necessary. This can take place in analogy to the removal, described above, of the heat of reaction by heating an external circulation stream or by internal heating in the reaction zone. In a suitable embodiment, the heat of reaction from the first or a preceding reaction zone can be used to control the temperature in the second or a subsequent reaction zone.

A further possibility for heating the precursors is to use the heat of reaction removed from the reaction mixture. In a specific configuration of the method, a reactor cascade composed of two reactors connected in series is employed, with the reaction in the second reactor being carried out adiabatically. This term is understood in the context of the present invention in the industrial and not in the physicochemical sense. Thus, when the reaction mixture flows through the second reactor it experiences an increase in temperature owing to the exothermic hydrogenation reaction. An adiabatic reaction process means a procedure in which the amount of heat released in the hydrogenation is taken up by the reaction mixture in the reactor, and no cooling by cooling devices is applied. The heat of reaction is thus removed with the reaction mixture from the second reactor, apart from a residual fraction which is given up to the surroundings through natural conduction and radiation of heat from the reactor.

To reduce the temperature gradient over all the reaction zones on use of an external cooling, the feed stream of the after-reactors can be drawn off after the external heat exchanger. The entry temperature of the after-reactor is thus reduced to the exit temperature of the heat exchanger and will be below the exit temperature of the main reactor. The exit temperature of the subsequent reaction zone is thus reduced.

In one embodiment, an additional mixing can take place in at least one of the reaction zones employed or in the reactor system as a whole. Additional mixing is particularly advantageous when the hydrogenation takes place with long holdup times of the reaction mixture. It is possible to use for the mixing for example the streams fed into the reaction zone by introducing them via suitable mixing devices such as nozzles into the respective reaction zone. It is also possible to employ for the mixing streams from the respective reaction zone which are guided in an external circulation. In a specific embodiment, the reactor system has a gas space from which a gaseous stream is taken and, if appropriate after controlling the temperature in a heat exchanger, metered back via a suitable mixing device, preferably a nozzle, into the liquid reaction mixture (circulating gas method). The circulating gas is sucked out of the gas space preferably by the mixing device, which is designed in the form of an ejector.

The hydrogen required for the hydrogenation can be fed into the first and additionally into the subsequent reaction zone(s). Hydrogen is preferably fed only into the first reaction zone.

The discharge from the hydrogenation can be subjected, before the enantiomeric enrichment, to a single-stage or multistage separation operation resulting at least in a stream comprising the major amount of the hydrogenation product and, if appropriate, additionally a stream comprising the hydrogenation catalyst. For this, the discharge from the hydrogenation can initially be subjected to a degassing to isolate excess hydrogen. The resulting liquid phase, which comprises the hydrogenation product, the catalyst and, if appropriate, solvent employed, can then be further fractionated by conventional methods known to the skilled worker. These include thermal fractionation by distillation or extractive fractionation.

For further working up, the enantiomer mixture obtained from the hydrogenation is subjected to an enantiomer-enriching crystallization with addition of a basic salt former. Suitable basic salt formers are customary asymmetric amines known to the skilled worker, such as, for example, (R)-phenethylamine. The ee values achieved on use of such asymmetric amines are usually about 99.5%. It has surprisingly been found that achiral basic compounds can also be employed as salt formers for the enantiomer-enriching crystallization. These are preferably selected from ammonia, primary amines such as methylamine, ethylamine, n-propylamine, isopropylamine, n-butylamine, n-pentylamine, n-hexylamine, cyclohexylamine, alkali metal hydroxides such as KOH, NaOH, LiOH, and alkaline earth metal hydroxides such as Ca(OH)₂ and Mg(OH)₂.

The enantiomer-enriching crystallization preferably takes place from a solvent which is selected from organic solvents, preferably water-miscible organic solvents, solvent mixtures, and mixtures of water-miscible organic solvents and water. Suitable organic solvents are monohydric alcohols such as methanol, ethanol, n-propanol, isopropanol, n-butanol, cyclohexanol; polyols such as ethylene glycol and glycerol; ethers and glycol ethers such as diethyl ether, dibutyl ether, anisole, dioxane, tetrahydrofuran, mono-, di-, tri- and polyalkylene glycol ethers; ketones such as acetone, butanone, cyclohexanone; mixtures of the aforementioned solvents, and mixtures of one or more of the aforementioned solvents with water. Solvents particularly preferably employed are alkanols and alkanol-water mixtures and specifically isopropanol and isopropanol-water mixtures.

In a suitable procedure, the product of the enantiomer-enriching hydrogenation can be dissolved or suspended in the solvent and then the salt former can be added as solution in the same or a different solvent or in solid form. Thus, it is possible for example to dissolve the product of the hydrogenation in an amount of solvent sufficient for complete dissolution, and then to add an aqueous solution of the salt former. In a preferred embodiment, the hydrogenation product is dissolved in isopropanol, and then an aqueous ammonia solution is added. A 20 to 30% strength aqueous ammonia solution is suitable for example. In a further preferred embodiment, the hydrogenation product is dissolved in isopropanol, and solid LiOH is added, and the resulting suspension is then stirred. An adequate stirring time is for example in the range from about 10 minutes to 12 hours, preferably 20 minutes to 6 hours, in particular 30 minutes to 3 hours.

The temperature in the enantiomer-enriching crystallization is generally in the range between the melting point and boiling point of the solvent or solvent mixture employed. In a suitable embodiment, the temperature can be increased and/or decreased one or more times during the crystallization in order to initiate the formation of crystals and/or to complete the precipitation of the desired enantiomer.

The solid isolated after the enantiomer-enriching crystallization advantageously has an ee of at least 98%, particularly preferably at least 99% and especially more than 99.5%.

The compounds isolated in the enantiomer-enriching crystallization may if desired be subjected to a protonation or a cation exchange. Thus, it is possible for example to bring the product of the crystallization into contact with a suitable acid, preferably a mineral acid such as HCl, H₂SO₄, H₃PO₄ for the protonation to result in an optically active compound of the formula I in which A is hydrogen. In a suitable procedure, the product of the crystallization is dissolved or suspended in water and then the pH is adjusted to about 0 to 4, preferably about 1, by adding acid. The free acid can be isolated by extracting the acidified solution or suspension with a suitable organic solvent, e.g. an ether such as methyl butyl ether, a hydrocarbon or hydrocarbon mixture, e.g. an alkane such as pentane, hexane, heptane, or an alkane mixture, ligroin or petroleum ether, or aromatic compounds such as toluene. Toluene is a preferred extractant. The acid can be obtained virtually quantitatively in this procedure, with the ee also being retained.

In a preferred embodiment, the method of the invention makes it possible to prepare optically active compounds of the formula I with the following absolute configuration

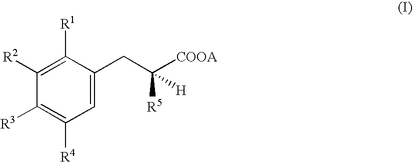

where R¹ to R⁵ and A have the aforementioned meanings. The method of the invention is thus particularly advantageously suitable for preparing intermediates which are suitable for further processing to synthon A and synthon A derivatives.

The invention therefore further relates to a method for preparing optically active compounds of the general formula III

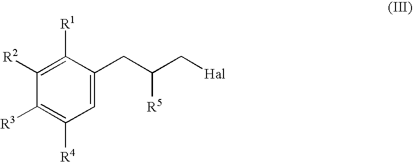

in which R¹ to R⁵ have the aforementioned meanings, and Hal is Cl, Br or I, in which
a compound of the general formula I as defined above is converted, in the case where A is not a metal cation or proton, by protonation into the acid,
the acid, obtained if appropriate after protonation, or a metal salt thereof is subjected to a reduction to obtain an alcohol of the general formula IV

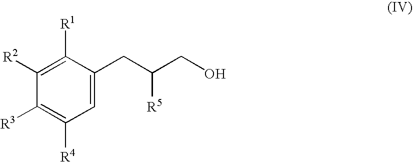

in which R¹ to R⁵ have the aforementioned meanings, and
the alcohol of the formula IV is subjected to a halodehydroxylation to obtain the optically active compound of the formula III.

The compound of the formula I is preferably employed in the form of the free acid for the reduction. The method for converting compounds of the formula I in which A is a cation equivalent different from protons into the free acid can be as described previously. For this purpose, preferably the compound of the formula I is brought into contact with a mineral acid such as HCl, H₂SO₄ or H₃PO₄. Protonation of the compound of the formula I preferably takes place in an aqueous medium. The free acid is preferably isolated using a suitable organic solvent, preferably by extraction with a water-immiscible or only slightly water-miscible solvent. Examples of suitable solvents are ethers such as diethyl ether, methyl butyl ether and methyl tert-butyl ether, the aforementioned hydrocarbons or hydrocarbon mixtures, aromatic compounds such as toluene, and halogenated aromatic compounds such as dichloromethane, chloroform, tetrachloromethane and 1,2-dichloroethane. The acid is preferably isolated and/or purified by extraction of an organic phase comprising the acid with an aqueous phase. It is possible with such a procedure, as described previously, to obtain the acid virtually quantitatively, with the ee likewise being retained.

Reagents suitable in principle for reducing compounds of the formula I in which A is a proton or a metal cation are those customary for reducing carboxylic acids to alcohols, such as complex hydrides, and catalytic hydrogenation methods with molecular hydrogen. Suitable methods and reaction conditions are described in J. March, Advanced Organic Chemistry, 4^{th} edition, published by John Wiley & Sons (1992), p. 1212 and Table 19.5, p. 1208, to which reference is made here. Complex hydrides such as LiAlH₄, AlH₃, LiAlH(OCH₃)₃, LiAlH(O-t-C₄H₉)₃, (i-C₄H₉)₂AlH (=DIBALH), NaAl(C₂H₅)₂H₂, NaAl(CH₃OC₂H₄O)₂H₂ (=Vitride), etc., are preferably employed.

Conversion of the alcohol of the general formula IV obtained in the reduction into an alkyl halide can take place by customary methods known to the skilled worker. Suitable methods are described in J. March, Advanced Organic Chemistry, 4^{th} edition, published by John Wiley & Sons (1992), pp. 431-433, to which reference is made here. A hydrohalic acid such as HCl, HBr, HI or an inorganic acid halide such as SOCl₂, PCl₅, PCl₃, POCl₃, etc., is preferably employed for the halodehydroxylation. The alcohol is preferably converted into the corresponding alkyl chloride (Hal=Cl). The latter is, in a particularly preferred embodiment of the method of the invention, synthon A.

The compound of the formula III can if desired be subjected to a final purification by customary methods known to the skilled worker, e.g. by recrystallization from a suitable solvent.

The method of the invention can advantageously be employed as part of an overall synthesis to prepare synthon A and synthon A derivatives. The invention therefore also relates to a method as defined above in which
a) an aromatic aldehyde of the general formula V

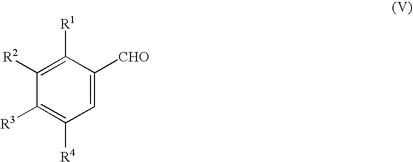

in which R¹ to R⁴ have the aforementioned meanings, is reacted with a carboxylic ester of the general formula VI

R⁵—CH₂—COOR⁷ (VI)
in which R⁵ has the meanings indicated in claim 1, and R⁷ is alkyl, cycloalkyl, aryl or alkylaryl, to obtain compounds of the general formula VII

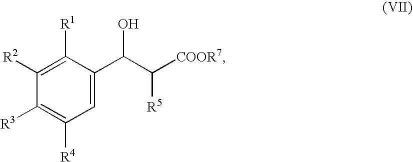

b) the hydroxyl group in the compounds of the formula VII is converted into a better leaving group and subjected to an elimination to obtain compounds of the general formula VIII

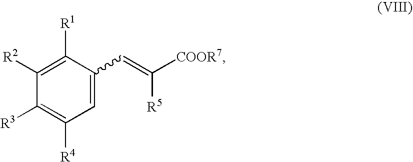

c) the compounds of the formula VIII are subjected to an ester hydrolysis to obtain compounds of the general formula II

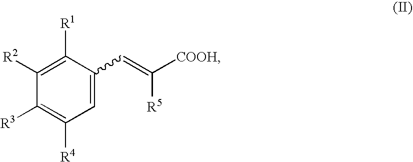

d) the compounds of the formula II are subjected to an enantioselective hydrogenation in the presence of a chiral hydrogenation catalyst to obtain a mixture of enantiomers enriched in one enantiomer,
e) the mixture of enantiomers obtained in the hydrogenation in step d) is subjected, for farther enantiomer enrichment, to a crystallization by adding a basic salt former in a solvent, and the solid which is formed thereby and is enriched in one stereoisomer is isolated,
f) if appropriate the isomer isolated in step e) is subjected to a protonation or a cation exchange to obtain the optically active compound of the formula I,
g) in the case where the radical A in the compound of the formula I is a cation equivalent different from hydrogen and metal cations, this equivalent is subjected to a protonation,
h) the acid or the metal salt thereof is subjected to a reduction to obtain an alcohol of the general formula IV

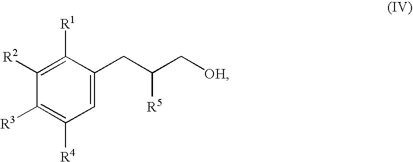

and
i) the alcohol of the formula IV is subjected to a halodehydroxylation to obtain the optically active compound of the formula III

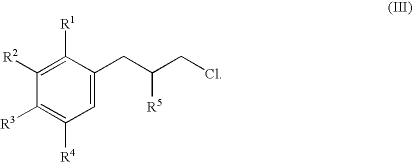

The optically active compounds of the general formula I obtained as intermediates in the method of the invention

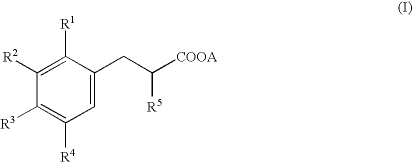

in which R¹ to R⁵ have the aforementioned meaning, and A is a cation derived from ammonia, primary amines, alkali metals and alkaline earth metals, are novel and the invention likewise relates thereto. The radical R⁵ in the compounds of the formula I is preferably a branched C₃-C₈-alkyl radical and in particular isopropyl. The compounds of the invention preferably have the following formula:

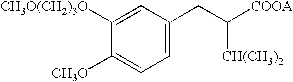

The compounds are in particular ones in which A is NH₄⁺ or Li⁺.

The aromatic aldehydes of the formula V employed as precursor in step a) are commercially available or can be prepared by customary methods known to the skilled worker. A suitable embodiment for preparing “synthon A” can start for example from 3-hydroxy-4-methoxybenzaldehyde(isovanillin) and subject the hydroxy function to an etherification to obtain 3-(3-methoxypropoxy)-4-methoxybenzaldehyde as compound of the formula V.

Suitable conditions for methods for reacting aromatic aldehydes with carboxylic esters which have acidic hydrogen atoms in the sense of an aldol reaction are described for example in J. March, Advanced Organic Chemistry, 4^{th} edition, published by John Wiley & Sons (1992), pp. 944-951, to which reference is made here. The reaction generally takes place in the presence of a strong base, which is preferably selected from alkali metal alcoholates such as sodium methanolate, potassium methanolate, potassium tert-butanolate, alkali metal hydrides such as sodium hydride, secondary amides such as lithium amide, lithium diisopropylamide, etc. The reaction preferably takes place at a temperature in the range from −80 to +30° C., in particular from −60 to +20° C. Examples of suitable solvents are ethers such as diethyl ether, tetrahydrofuran and dioxane, aromatic compounds such as benzene, toluene and xylene, etc.

The dehydration in reaction step b) is likewise known in principle. The hydroxyl group is converted into a better leaving group preferably by reaction with a sulfonic acid or a derivative thereof, such as benzenesulfonic acid, toluenesulfonic acid, methylsulfonic acid, trifluoromethylsulfonic acid or a derivative, e.g. a halide, thereof. In a preferred embodiment, the dehydration takes place in a solvent able to form low-boiling azeotropes with water, such as benzene or, preferably, toluene. The water formed in the reaction can then be removed by azeotropic distillation (with water trap) by customary methods known to the skilled worker. It is possible in this procedure to employ the acid able to form the leaving group merely in catalytic amounts. It has been found that this procedure advantageously results in cis/trans isomer mixtures of compounds of the formula VIII which comprise the cis isomer in excess.

Methods for hydrolyzing carboxylic esters (step c)) to the corresponding carboxylic acids or to salts thereof are likewise known in principle and are described for example in J. March, Advanced Organic Chemistry, 4^{th} edition, published by John Wiley & Sons (1992), pp. 378-383, to which reference is made here. In principle, acid or basic ester hydrolysis is possible.

Concerning steps d) to i) of the method, reference is made to previous statements about suitable and preferred conditions for the methods in their entirety.

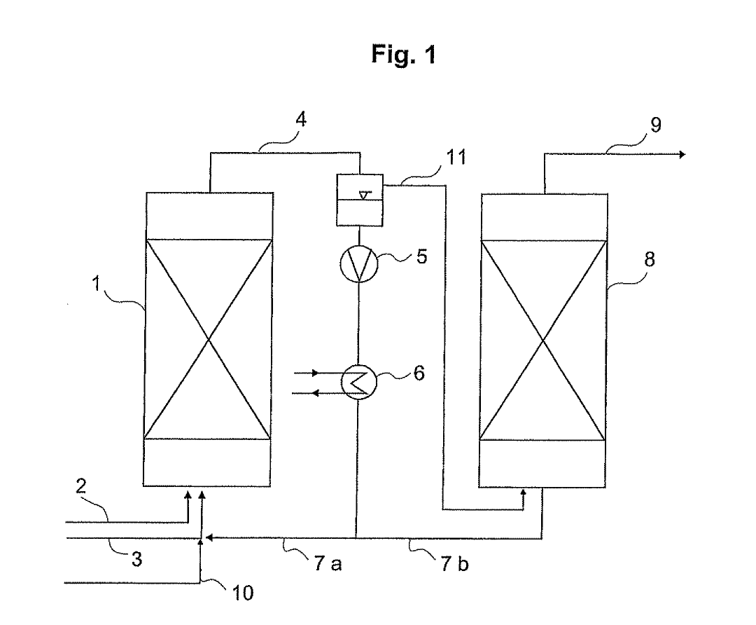

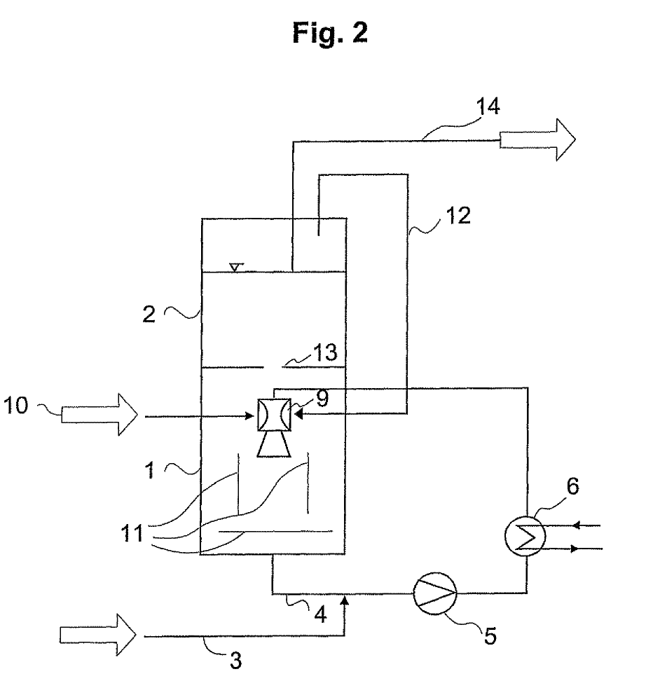

Advantageous configurations of the hydrogenation step of the method of the invention in relation to a continuous reaction process are depicted in FIGS. 1 and 2 and are explained hereinafter.

FIG. 1 shows the diagram of a two-stage reactor cascade suitable for carrying out the hydrogenation method, dispensing, for reasons of clarity, with the representation of details irrelevant to explanation of the invention. The system comprises a first hydrogenation reactor (1) and a second hydrogenation reactor (8). The hydrogenation reactor (1) is designed as circulating reactor and the hydrogenation reactor (8) is designed as adiabatic flow tube reactor. Hydrogen gas is passed through the pipeline (2) under pressure into the reactor (1), and a solution of the compound to be hydrogenated is passed into the reactor (1) through the pipeline (3). If the catalyst is not present in the precursor solution, it is fed via a further line (10) either directly to the reactor or upstream of the circulating pump. A discharge is taken from the reactor (1) through the pipeline (4) and the pump (5), cooled in the heat exchanger (6) and divided into two part-streams (7a) and (7b). The part-stream (7a) is returned to the reactor (1) as recycle stream. The characteristic holdup time distribution in the reactor (1) depends substantially on the circulated stream (7a). The second part-stream is fed through the pipeline (7b) to the reactor (8) to complete the hydrogenation. The discharge stream (4) may comprise dissolved or gaseous fractions for example of hydrogen. In an alternative embodiment, the stream (4) is fed to a phase-separation tank, and the gaseous fractions are fed to the reactor (8) via the separate line (11). In a further alternative embodiment, the reactor (8) is charged with hydrogen not through a gaseous feed taken from the reactor (1) but with fresh hydrogen through a separate feed line. The hydrogenation product leaves the reactor (8) through the pipeline (9).

FIG. 2 shows the diagram of a reactor suitable for carrying out the hydrogenation process and composed of two hydrogenation compartments, dispensing once again, for reasons of clarity, with representation of details irrelevant to explanation of the invention. The reactor comprises two hydrogenation compartments (1) and (2), both designed for back-mixing. Compartment (1) is designed as jet loop reactor. The hydrogenation takes place in compartment (2) under quasi-adiabatic conditions. A discharge stream is taken from compartment (1) via circulating pump (5) and fed together with fed-in hydrogen gas (3) through heat exchanger (6) to the flow-controlled nozzle (9). It is possible if necessary to feed hydrogen gas via feed line (10) to the nozzle (9). The ejection stream of the nozzle (9) is limited by the deflection plates (11). The after reactor (2) is fed through a perforated plate with at least one orifice (13). To improve mixing, a gas circulation (12) can be employed with use of an ejector (9). The hydrogenation product is taken from the liquid space of the compartment (2) through pipeline (14).

The invention is explained by means of the following non-restrictive examples.

### EXAMPLES

### Example 1

Preparation of

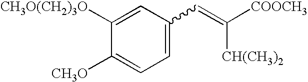

544 ml of a 15% strength solution of n-butyllithium in hexane, 98.2 g of methyl isovalerate in 45 ml of tetrahydrofuran and 170 g of 4-methoxy-3-(3-methoxypropyloxy)benzaldehyde in 75 ml of tetrahydrofuran were added dropwise to a solution of 88.5 g of diisopropylamine in 300 ml of tetrahydrofuran at −50° C. The resulting solution was allowed to warm to room temperature over the course of 2 h and was then stirred at this temperature for 1 h. Subsequently, 300 ml of water were added dropwise to the reaction solution, the pH was adjusted to 1 with concentrated HCl, the phases were separated and the aqueous phase was then extracted twice with 300 ml of toluene. The organic phases were combined and the solvent was evaporated off in a rotary evaporator. The residue was taken up in 500 ml of toluene and, after addition of 6 g of p-toluenesulfonic acid, heated under reflux with a water trap for 3.5 h. The reaction mixture was washed with 150 ml of saturated NaHCO₃ solution and 300 ml of water and dried over sodium sulfate, and the solvent was stripped off in a rotary evaporator. 242 g of product were obtained.

The reaction product was analyzed by the following HPLC method:

Column: Waters Symmetry C18 5 μm, 250×4.6 mm

Eluent: A) 0.1 vol % H₃PO₄ in water, B) 0.1 vol % H₃PO₄ in CH₃CN

Gradient (based on eluent B): 0 min (35%) 20 min (100%) 30 min (100%) 32 min (35%)

Flow rate: 1 ml/min, temperature: 20° C., volume injected: 5 μl

Detection: UV detector at 205 nm, BW=4 nm

In this method, the cis ester eluted at 15.7 min, the trans ester at 16.2 min, the cis acid at 10.6 min, the trans acid at 10.9 min and the aromatic aldehyde employed as precursor at 7.9 min.

The resulting product comprised 69.1% cis ester, 21.0% trans ester, 0.8% aldehyde, remaining components not assigned (area % of the HPLC peaks).

Hydrolysis of the resulting ester mixture is possible by customary methods, for example with KOH in an ethanol/water mixture.

### Example 2

Preparation of

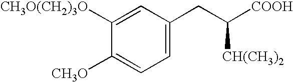

30.1 g of the cis/trans acid mixture obtained after ester hydrolysis were introduced into 55.4 g of methanol under a protective gas atmosphere in a 300 ml steel autoclave. Addition of 2.05 mg of (R)-phanephos-Rh-(COD)BF₄×1 (C₂H₅)₂O was followed by hydrogenation under a hydrogen pressure of 200 bar and at a temperature of 100° C. for 12 h. The hydrogenation was quantitative after 12 h. The enantiomeric excess of the product was 83%.

Analysis both of the product of the hydrogenation and of the subsequent crystallization (Examples 3 and 4) took place by the following HPLC method:

Column: CHIRALPAK AD-H (250×4.6 mm)

Fluent: mixture of 950 ml of n-heptane, 50 ml of ethanol and 2 ml of trifluoroacetic acid

Flow rate: 1.0 ml/min, column temperature 25° C., volume injected 25 μl

Detection: UV detector at 225 nm

In this method, the cis isomer (precursor) eluted at 22.3 min, the trans isomer (precursor) at 30.7 min, the (S) enantiomer (product) at 11.7 min and the (R) enantiomer (product) at 14.0 min.

### Example 3

Enantiomeric Enrichment by Crystallization With Ammonia

95.6 g of a crude hydrogenation product obtained in Example 2 were dissolved in 750 ml of isopropanol, and 44.2 ml of 25% strength ammonia solution were added with stirring. Crystal formation was observable after 10 min. After subsequent stirring at room temperature for 3 h, the crystal/solution was cooled to −10° C. and the crystals were isolated by filtration. The resulting solid was washed twice with 100 ml of cold petroleum ether and dried in a drying oven at 30° C. overnight.

The ammonium salt was obtained in a yield of 78% based on the crude product employed with an ee of 98.9%.

### Example 4

Enantiomeric Enrichment by Crystallization With LiOH

0.5 g of a crude hydrogenation product obtained in Example 2 was dissolved in 5 ml of isopropanol, 40 mg of LiOH were added, and the resulting suspension was stirred at room temperature for 1 h. The resulting crystals were isolated by filtration and the solid was washed twice with 2 ml of cold petroleum ether and dried in a drying oven at 30° C. overnight. 0.3 g of crystals (60%) with an ee of 97.5% were obtained.

### Example 5

Preparation of Synthon A Acid

The ammonium salt obtained in Example 3 was dissolved in 500 ml of water, and the pH was adjusted to a value of 1 by adding 30 ml of cone. HCl. The aqueous phase was extracted twice with 250 ml of toluene each time, the combined organic phases were washed with deionized water, and then the solvent was concentrated to 150 ml in a rotary evaporator. Crystal formation was observed after stirring at room temperature for 10 minutes. After subsequent stirring at room temperature for 3 h, the crystal solution was cooled to −10° C. and the crystals were isolated by filtration. The resulting solid was washed twice with 100 ml of cold petroleum ether each time and dried in a drying oven at 30° C. overnight. 69.3 g of synthon A acid were obtained as a white solid in a yield of 99% and with an ee of 98.9%.

### Example 6

### Scale-Up of Example 1

68.4 kg of diisopropylamine and 155 kg of tetrahydrofuran (THF) were introduced into a 1 m³ stainless steel vessel and cooled to −50° C. This was followed by successive metering in of 274 kg of a 15% strength solution of n-butyllithium in hexane, 72.7 kg of mnethiyl isovalerate, 30 kg of THF, and 139 kg of 4-methoxy-3-(3-methoxypropyloxy)benzaldehyde followed by 30 kg of THF, during which the temperature was kept below −30° C. After completion of the addition, the reactor was warmed to 20° C. at 10 K/h. 500 l of deionized water were introduced into a 2.5 m³ steel/enamel vessel, the contents of the stainless steel vessel were fed in at 20° C., and the stainless steel vessel was rinsed with 88 kg of THF. The pH was then adjusted to 1 by adding 200 kg of 31% strength HCl, and the phases were separated. The upper organic phase was evacuated stepwise in a 1 m³ steel/enamel vessel to 400 mbar, and the THF was distilled out. Addition of 585 kg of toluene and 5.4 kg of p-toluenesulfonic acid in 12 l of deionized water was followed by azeotropic distillation of toluene/water from the contents of the vessel until the distillate was pure toluene. After cooling to 20° C., the contents of the vessel were washed with 200 l of saturated NaHCO₃ solution and 200 l of water, and the organic phase was employed directly in Example 7. The crude 28% strength product solution comprised 160 kg of cis-trans isomer mixture (3.2:1).

### Example 7

### Hydrolysis of the Compound Prepared in Example 6

The product solutions from two batches of the previous stage (Example 6) were combined in a 2 m³ stainless steel vessel, and most of the toluene was distilled off under a pressure of 150 mbar. 720 kg of 25% strength NaOH were fed in at an internal temperature of 80° C., and distillation was carried out for 6 h until the internal temperature reached 115° C. The contents of the vessel were cooled to 60° C. and left to settle for phase separation. Removal of 500 l of a clear aqueous phase was followed by addition of 630 kg of water and 300 kg of toluene to the brown organic phase in the vessel and by stirring at 60° C. for 30 minutes. Subsequently, 1100 l of an aqueous phase were discharged and the organic phase was discarded. The aqueous phase was extracted a second time with 300 kg of toluene. The aqueous phase was then mixed in a 2.5 m³ steel/enamel vessel with 590 kg of toluene, acidified by adding 105 kg of 75% strength sulfuric acid, and stirred for 30 minutes. The phases were separated and the aqueous phase was again extracted with 590 kg of toluene. The organic phases were combined and washed with 700 kg of deionized water. The washed organic phase was heated to boiling under 150 mbar, and the toluene was distilled out until the bottom temperature was 120° C. The bottom product was diluted by adding 350 kg of methanol. 302 kg of acid were obtained as cis-trans isomer mixture (3.2:1).

### Example 8

### Scale-Up of Example 2

486 kg of the cis/trans acid mixture obtained in analogy to Example 7 were introduced into 1118 kg of methanol in a 3.5 m³ steel autoclave under a protective gas atmosphere. Addition of a methanolic solution of 64.3 g of (R)-phanephos-Rh-(COD)BF₄ was followed by hydrogenation under a hydrogen pressure of 200 bar and at a temperature of 75° C. The hydrogenation was quantitative after 14 h. The enantiomeric excess of the product was 86%.

### Example 9

### Scale-Up of Examples 3 and 5

A 2 m³ stainless steel vessel was charged with 1000 kg of a 25% strength solution of the hydrogenation product from Example 8 stage, and most of the methanol was distilled out under a pressure of 600 mbar. 1000 kg of isopropanol were added to the bottom product and, at 50° C., 57 kg of a 25% strength aqueous ammonia solution were added. After completion of the addition, the mixture was stirred at 50° C. for 30 minutes, then cooled at 10 K/h to 0° C. and stirred at 0° C. for 1 h. The mass of crystals was centrifuged in 4 portions in a peeler centrifuge, and the crystals were washed with in each case 100 kg of isopropanol and discharged with a residual moisture content of about 60%.

The crystals were dissolved in 800 kg of water in a 2 m³ steel-enamel vessel and covered with 400 kg of toluene. At 30° C., 120 l of a 31% strength HCl solution were added and the mixture was stirred for 30 minutes. After phase separation, the aqueous phase was again extracted with 400 kg of toluene, and the organic phases were combined and washed with 300 kg of deionized water. 500 l of toluene were distilled out under atmospheric pressure. 205 kg of synthon A acid as a 28% strength solution in toluene were obtained with an ee of 99.2%.

### Example 10

Preparation of

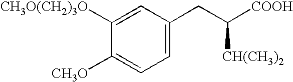

by hydrogenation with phanephos under 80 bar.

30 g of the cis/trans acid mixture obtained after ester hydrolysis according to Example 1 were introduced into 59 g of methanol in a 300 ml steel autoclave under a protective gas atmosphere. Addition of 3.8 mg of (R)-phanephos-Rh-(COD)BF₄ was followed by hydrogenation under a hydrogen pressure of 80 bar and at a temperature of 90° C. The hydrogenation was quantitative after 20 h. The enantiomeric excess of the product was 83%.

### Example 11

Preparation of

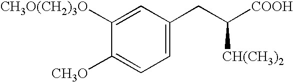

by hydrogenation with (ligand C)

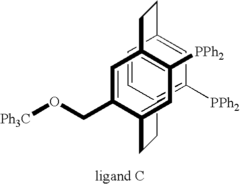

30 g of the cis/trans acid mixture obtained after ester hydrolysis in analogy to Example 1 were introduced into 62 g of methanol in a 300 ml steel autoclave under a protective gas atmosphere. Addition of 5.0 mg of (R)-(ligand C)-Rh-(NBD)BF₄ was followed by hydrogenation under a hydrogen pressure of 80 bar and at a temperature of 90° C. The hydrogenation was quantitative after 20 h. The enantiomeric excess of the product was 83%.

### Example 12

Preparation of

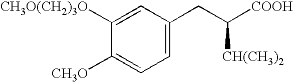

by hydrogenation with ligand D

30 g of the cis/trans acid mixture obtained after ester hydrolysis in analogy to Example 1 were introduced into 60 g of methanol in a 300 ml steel autoclave under a protective gas atmosphere. Addition of 4.7 mg of (ligand D)-Rh-(COD)BF₄ was followed by hydrogenation under a hydrogen pressure of 200 bar and at a temperature of 100° C. The hydrogenation was quantitative after 8 h. The enantiomeric excess of the product was 83%.

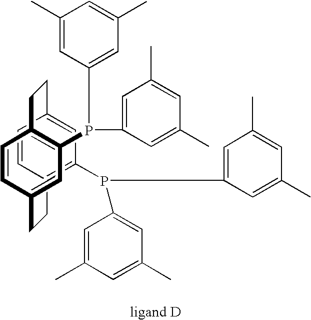

### Example 13

Preparation of

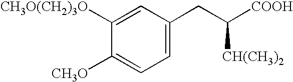

by hydrogenation with ligand E

40 g of the cis/trans acid mixture obtained after ester hydrolysis in analogy to Example 1 were introduced into 40 g of methanol in a 300 ml steel autoclave under a protective gas atmosphere. Addition of 6.0 mg of (ligand E)-Rh-(COD)BF₄ was followed by hydrogenation under a hydrogen pressure of 80 bar and at a temperature of 90° C. The hydrogenation was quantitative after 12 h. The enantiomeric excess of the product was 80%.

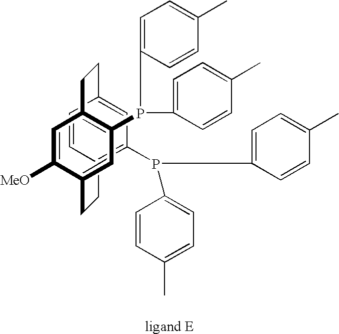

### Example 14

Preparation of

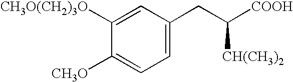

by hydrogenation with ligand F

40 g of the cis/trans acid mixture obtained after ester hydrolysis in analogy to Example 1 were introduced into 40 g of methanol in a 300 ml steel autoclave under a protective gas atmosphere. Addition of 5.9 mg of (ligand F)-Rh-(COD)BF₄ (as methanolic solution) was followed by hydrogenation under a hydrogen pressure of 80 bar and at a temperature of 90° C. The hydrogenation was quantitative after 12 h. The enantiomeric excess of the product was 82%.

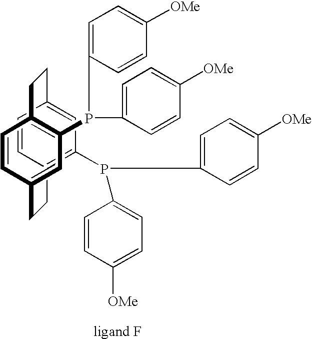

### Example 15

Preparation of

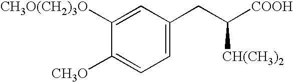

by hydrogenation with ligand G

40 g of the cis/trans acid mixture obtained after ester hydrolysis in analogy to Example 1 were introduced into 40 g of methanol in a 300 ml steel autoclave under a protective gas atmosphere. Addition of 5.3 mg of (ligand G)-Rh-(COD)BF₄ was followed by hydrogenation under a hydrogen pressure of 80 bar and at a temperature of 90° C. The hydrogenation was quantitative after 16 h. The enantiomeric excess of the product was 81%.

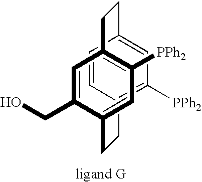

### Example 16

Preparation of

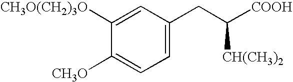

by hydrogenation with ligand H

40 g of the cis/trans acid mixture obtained after ester hydrolysis in analogy to Example 1 were introduced into 40 g of methanol in a 300 ml steel autoclave under a protective gas atmosphere. Addition of 5.5 mg of (ligand H)-Rh-(COD)BF₄ was followed by hydrogenation under a hydrogen pressure of 80 bar and at a temperature of 90° C. The hydrogenation was quantitative after 16 h. The enantiomeric excess of the product was 82%.

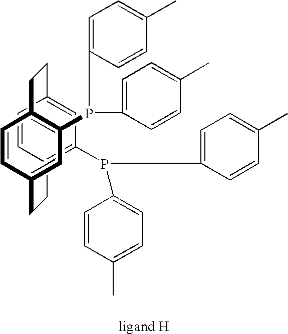

### Example 17

Recrystallization of Crude Synthon A

200 kg of a synthon A crude product with a content of 89.1% by weight (detennined by HPLC) and an ee of 97.2% were mixed at 50° C. with 400 kg of methanol in a 1 m³ steel-enamel vessel and cooled to 30° C. Seeding with crystals of pure synthon A was followed by cooling at a rate of 10 K/h to −10° C., and the resulting mass of crystals was filtered off on a process filter, washed with about 100 kg of cold methanol and dried in vacuo. 144 kg of synthon A were obtained as white crystals with a content of 99.5% by weight. The enantiomeric excess was 99.8%.

1-21. (canceled)

22. A method for preparing optically active compounds of formula (I) 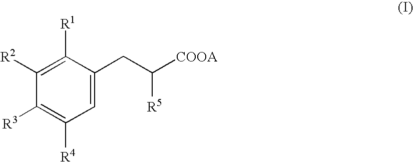
wherein
R¹, R², R³, and R⁴
are, independently of one another, hydrogen, C₁-C₆-alkyl, halo-C₁-C₆-alkyl, hydroxy-C₁-C₆-alkyl, C₁-C₆-alkoxy, hydroxy-C₁-C₆-alkoxy, C₁-C₆-alkoxy-C₁-C₆-alkyl, hydroxy-C₁-C₆-alkoxy-C₁-C₆-alkyl, C₁-C₆-alkoxy-C₁-C₆-alkoxy, or hydroxy-C₁-C₆-alkoxy-C₁-C₆-alkoxy;
R⁵ is C₁-C₆-alkyl, C₅-C₈-cycloalkyl, phenyl, or benzyl; and
A is hydrogen or a cation equivalent; wherein
the cis isomer or a cis/trans isomer mixture of compounds of formula (II) 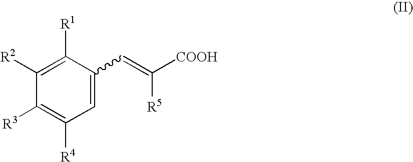
is subjected to an enantioselective hydrogenation in the presence of a chiral hydrogenation catalyst to obtain a mixture of enantiomers enriched in one enantiomer; wherein
the mixture of enantiomers obtained in the hydrogenation is subjected, for further enantiomer enrichment, to a crystallization by adding a basic salt former in a solvent, and the solid which is formed thereby and is enriched in one stereoisomer is isolated; and wherein
the isolated isomer is optionally subjected to a protonation or a cation exchange to obtain the optically active compound of formula (I).

23. The method of claim 22, wherein a cis/trans isomer mixture comprising at least 50% by weight of the cis isomer is employed for the hydrogenation.

24. The method of claim 22, wherein a cis/trans isomer mixture comprising at least 1% by weight of the trans isomer is employed for the hydrogenation.

25. The method of claim 22, wherein said chiral hydrogenation catalyst is a transition metal complex comprising at least one ligand of formula 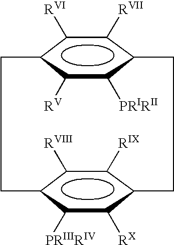
wherein
R^{I}, R^{II}, R^{III}, and R^{IV}
are, independently of one another, alkyl, cycloalkyl, heterocycloalkyl, aryl, or hetaryl;
R^{V}, R^{VI}, R^{VII}, R^{VIII}, R^{IX}, and R^{X}
are, independently of one another, hydrogen, alkyl, alkylene-OH, alkylene-NE¹E², alkylene-SH, alkylene-OSiE³E⁴, cycloalkyl, heterocycloalkyl, aryl, hetaryl, OH, SH, polyalkylene oxide, polyalkyleneimine, alkoxy, halogen, COOH, carboxylate, SO₃H, sulfonate, NE¹E², nitro, alkoxycarbonyl, acyl, or cyano; and
E¹, E², E³, and E⁴
are, identically or differently, hydrogen, alkyl, cycloalkyl, aryl, or alkylaryl.

26. The method of claim 25, wherein R^{I}, R^{II}, R^{III}, and R^{IV} are, independently of one another, phenyl, tolyl, methoxyphenyl, xylyl, or methoxyxylyl.

27. The method of claim 25, wherein one of R^{V}, R^{VI}, and R^{VII} and/or one of R^{VIII}, R^{IX}, and R^{X} are selected from the group consisting of C₁-C₆-alkyl, C₁-C₄-alkylene-OH, C₁-C₄-alkylene-OSi(C₁-C₄-alkyl)₂, C₁-C₄-alkoxy, C₁-C₄-alkylene-OC(alkyl)₃, and C₁-C₄-alkylene-OC(aryl)₃.

28. The method of claim 25, wherein said catalyst comprises at least one ligand selected from the compounds of formulae:

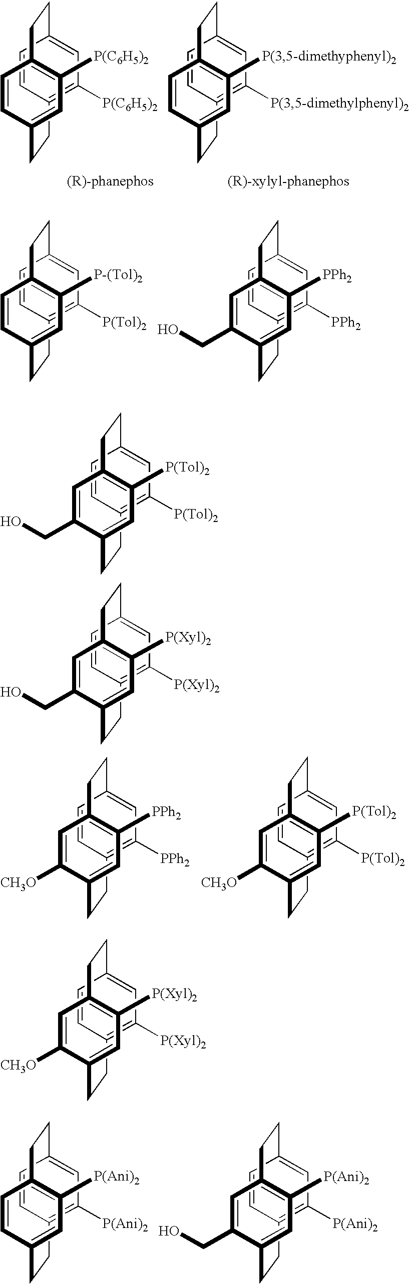

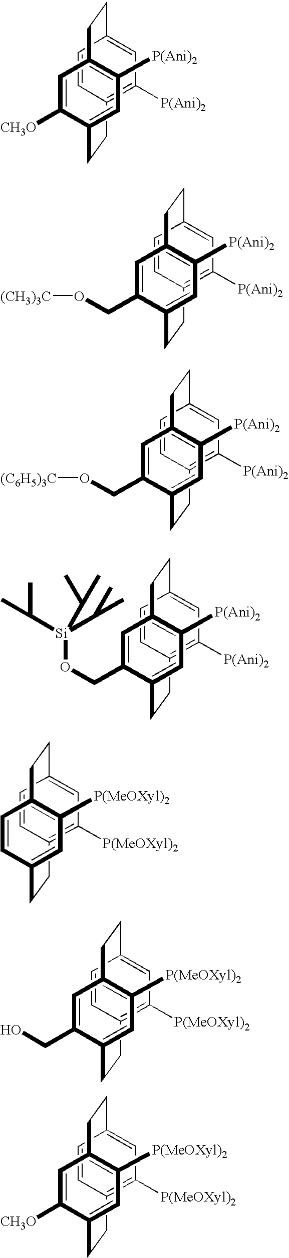

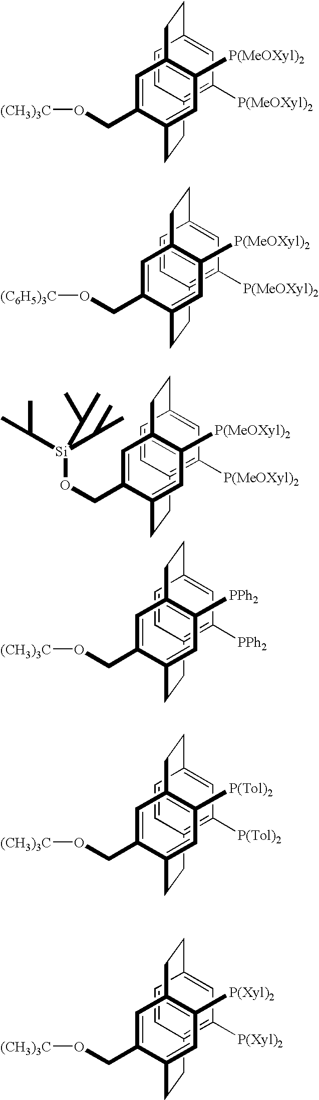

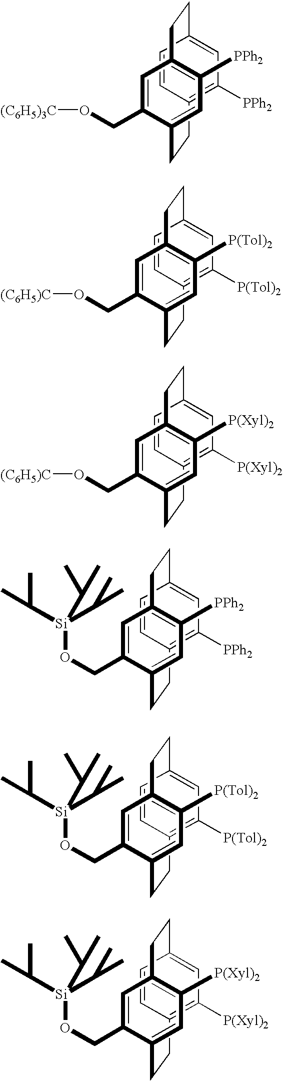

29. The method of claim 25, wherein said catalyst comprises at least one ligand selected from the compounds of formulae:

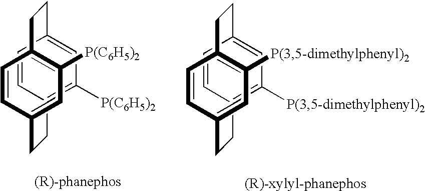

30. The method of claim 22, wherein said hydrogenation is performed continuously.

31. The method of claim 29, wherein
i) a mixture of isomers of compounds of formula (II) and hydrogen are fed into a first reaction zone and reacted in the presence of a chiral hydrogenation catalyst to partial conversion; and
ii) a stream is taken from said first reaction zone and hydrogenated in at least one additional reaction zone.

32. The method of claim 22, wherein said salt former is an achiral basic compound.

33. The method of claim 32, wherein said salt former is selected from the group consisting of ammonia, primary amines, alkali metal hydroxides, and alkaline earth metal hydroxides.

34. The method of claim 32, wherein said salt former is ammonia or LiOH and wherein isopropanol is employed as solvent for the crystallization.

35. The method of claim 22, wherein the solid isolated after the crystallization has an enantiomeric excess of at least 98%.

36. The method of claim 22, wherein an optically active compound is obtained having the formula 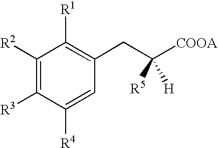
is obtained.

37. A method for preparing optically active compounds of formula (III) 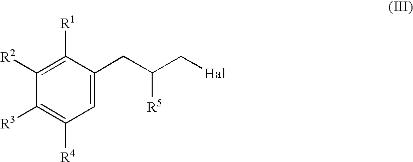
wherein
R¹, R², R³, and R⁴
are, independently of one another, hydrogen, C₁-C₆-alkyl, halo-C₁-C₆-alkyl, hydroxy-C₁-C₆-alkyl, C₁-C₆-alkoxy, hydroxy-C₁-C₆-alkoxy, C₁-C₆-alkoxy-C₁-C₆-alkyl, hydroxy-C₁-C₆-alkoxy-C₁-C₆-alkyl, C₁-C₈-alkoxy-C₁-C₆-alkoxy, or hydroxy-C₁-C₆-alkoxy-C₁-C₆-alkoxy;
R⁵ is C₁-C₆-alkyl, C₅-C₈-cycloalkyl, phenyl, or benzyl; and
Hal is Cl, Br, or I; wherein
the compound of claim 22 is converted, in the case where A is a cation equivalent different from hydrogen and metal cations, by protonation into the acid; wherein
the acid or the metal salt thereof is subjected to a reduction to obtain an alcohol of formula (IV) 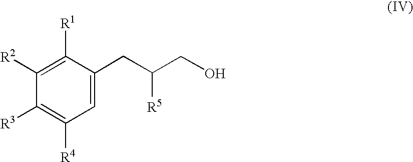
wherein
R¹, R², R³, and R⁴
are, independently of one another, hydrogen, C₁-C₆-alkyl, halo-C₁-C₆-alkyl, hydroxy-C₁-C₆-alkyl, C₁-C₆-alkoxy, hydroxy-C₁-C₆-alkoxy, C₁-C₆-alkoxy-C₁-C₆-alkyl, hydroxy-C₁-C₆-alkoxy-C₁-C₆-alkyl, C₁-C₆-alkoxy-C₁-C₆-alkoxy, or hydroxy-C₁-C₆-alkoxy-C₁-C₆-alkoxy;
R⁵ is C₁-C₆-alkyl, C₅-C₈-cycloalkyl, phenyl, or benzyl; and
said alcohol of formula (IV) is subjected to a halodehydroxylation to obtain the optically active compound of the formula III.

38. The method of claim 37, wherein
a) an aromatic aldehyde of formula (V) 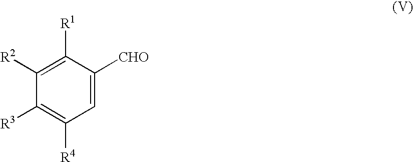
wherein
R¹, R², R³, and R⁴
are, independently of one another, hydrogen, C₁-C₆-alkyl, halo-C₁-C₆-alkyl, hydroxy-C₁-C₆-alkyl, C₁-C₆-alkoxy, hydroxy-C₁-C₆-alkoxy, C₁-C₆-alkoxy-C₁-C₆-alkyl, hydroxy-C₁-C₆-alkoxy-C₁-C₆-alkyl, C₁-C₆-alkoxy-C₁-C₆-alkoxy, or hydroxy-C₁-C₆-alkoxy-C₁-C₆-alkoxy;
is reacted with a carboxylic ester of formula (VI)
R⁵—CH₂—COOR⁷ (VI)
wherein
R⁵ is C₁-C₆-alkyl, C₅-C₈-cycloalkyl, phenyl, or benzyl; and
R⁷ is alkyl, cycloalkyl, aryl, or alkylaryl,
to obtain compounds of formula (VII) 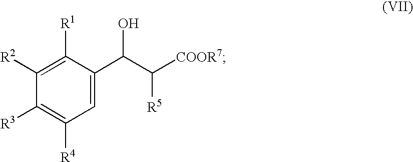
b) the hydroxyl group in the compounds of formula (VII) is converted into a better leaving group and subjected to an elimination to obtain compounds of formula (VIII) 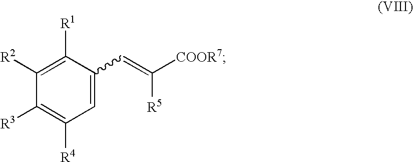
c) the compounds of formula (VIII) are subjected to an ester hydrolysis to obtain compounds of formula (II) 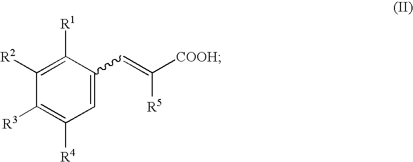
d) the compounds of formula (II) are subjected to an enantioselective hydrogenation in the presence of a chiral hydrogenation catalyst to obtain a mixture of enantiomers enriched in one enantiomer;
e) the mixture of enantiomers obtained in the hydrogenation in d) is subjected, for further enantiomer enrichment, to a crystallization by adding a basic salt former in a solvent, and the solid which is formed thereby and is enriched in one stereoisomer is isolated;
f) the isomer isolated in step e) is optionally subjected to a protonation or a cation exchange to obtain the optically active compound of formula (I);
g) where A is a cation equivalent different from hydrogen and metal cations, this equivalent is subjected to a protonation;
h) the acid or metal salt thereof is subjected to a reduction to obtain an alcohol of formula (IV) 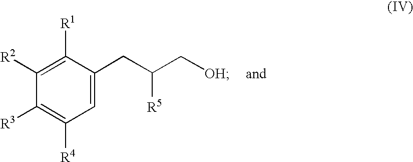
i) said alcohol of formula (IV) is subjected to a halodehydroxylation to obtain the optically active compound of formula (III).

39. An optically active compound of formula (I) 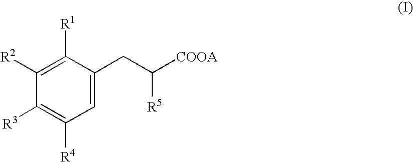
wherein
R¹, R², R³, and R⁴
are, independently of one another, hydrogen, C₁-C₆-alkyl, halo-C₁-C₆-alkyl, hydroxy-C₁-C₆-alkyl, C₁-C₆-alkoxy, hydroxy-C₁-C₆-alkoxy, C₁-C₆-alkoxy-C₁-C₆-alkyl, hydroxy-C₁-C₆-alkoxy-C₁-C₆-alkyl, C₁-C₆-alkoxy-C₁-C₆-alkoxy, or hydroxy-C₁-C₆-alkoxy-C₁-C₆-alkoxy;
R⁵ is C₁-C₆-alkyl, C₅-C₈-cycloalkyl, phenyl, or benzyl; and
A is a cation derived from ammonia, primary amines, alkali metals, and alkaline earth metals.

40. The compound of claim 39, wherein R⁵ is a branched C₃-C₈-alkyl radical.

41. The compound of claim 39, wherein said compound has the formula

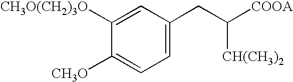

42. The compound of claim 39, wherein A is NH₄⁺ or Li⁺.
